(19) United States
(12) Patent Application Publication
(10) Pub. No.: US 2021/0293805 A1
(43) Pub. Date: Sep. 23, 2021

(54) MACHINE VISION TECHNIQUES, INCLUDING IMAGE PROCESSING TECHNIQUES, FOR DIAGNOSTIC TESTING

(71) Applicant: Detect, Inc., Guilford, CT (US)

(72) Inventors: Jonathan M. Rothberg, Guilford, CT (US); Benjamin Rosenbluth, Hamden, CT (US)

(73) Assignee: Detect, Inc., Guilford, CT (US)

(21) Appl. No.: 17/203,498

(22) Filed: Mar. 16, 2021

Related U.S. Application Data
(60) Provisional application No. 63/116,603, filed on Nov. 20, 2020.
(60) Provisional application No. 63/081,201, filed on Sep. 21, 2020.
(60) Provisional application No. 63/074,524, filed on Sep. 4, 2020.
(60) Provisional application No. 63/068,303, filed on Aug. 20, 2020.
(60) Provisional application No. 63/065,131, filed on Aug. 13, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/016,797, filed on Apr. 28, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. G01N 33/543; G01N 33/487; G01N 33/569; G01N 1/02

(52) U.S. Cl. CPC G01N 33/54386; G01N 33/48792; G01N 2001/028; G01N 1/02; G01N 33/56983

(57) ABSTRACT

Described herein in one embodiment is a computer-implemented method comprising accessing information representing a detection component of a diagnostic test and determining, based at least in part on the information representing the detection component of the diagnostic test, results of the diagnostic test. Described herein in one embodiment is a method of performing a diagnostic test on a subject. In one embodiment, the method comprises obtaining a sample from the subject, processing the sample, analyzing the sample with a detection component of the diagnostic test, and performing a computer-implemented method comprising accessing information representing the detection component of a diagnostic test, and determining, based at least in part on the information representing the detection component of the diagnostic test, results of the diagnostic test.

### RELATED APPLICATIONS

This application claims priority under 35 U.S.C. § 119(e) to U.S. Provisional Patent Application No. 62/991,039, titled “VIRAL RAPID TEST,” filed Mar. 17, 2020; U.S. Provisional Patent Application No. 63/002,209, titled “VIRAL RAPID TEST,” filed Mar. 30, 2020; U.S. Provisional Patent Application No. 63/010,578, titled “VIRAL RAPID TEST,” filed Apr. 15, 2020; U.S. Provisional Patent Application No. 63/010,626, titled “VIRAL RAPID COLORIMETRIC TEST,” filed Apr. 15, 2020; U.S. Provisional Patent Application No. 63/013,450, titled “METHOD OF MAKING AND USING A VIRAL TEST KIT,” filed Apr. 21, 2020; U.S. Provisional Patent Application No. 63/016,797, titled “SAMPLE SWAB WITH BUILD-IN ILLNESS TEST,” filed Apr. 28, 2020; U.S. Provisional Patent Application No. 63/022,534, titled “RAPID DIAGNOSTIC TEST,” filed May 10, 2020; 63/022,533, titled “RAPID DIAGNOSTIC TEST,” filed May 10, 2020; 63/036,887, titled “RAPID DIAGNOSTIC TEST,” filed Jun. 9, 2020; 63/074,524, titled “RAPID DIAGNOSTIC TEST WITH INTEGRATED SWAB,” filed Sep. 4, 2020; 63/081,201, titled “RAPID DIAGNOSTIC TEST,” filed Sep. 21, 2020; U.S. Provisional Patent Application No. 63/065,131, titled “APPARATUSES AND METHODS FOR PERFORMING RAPID DIAGNOSTIC TESTS,” filed Aug. 13, 2020; U.S. Provisional Patent Application No. 63/059,928, titled “RAPID DIAGNOSTIC TEST,” filed Jul. 31, 2020; U.S. Provisional Patent Application No. 63/068,303, titled “APPARATUSES AND METHODS FOR PERFORMING RAPID MULTIPLEXED DIAGNOSTIC TESTS,” filed Aug. 20, 2020; U.S. Provisional Patent Application No. 63/027,859, titled “RAPID SELF ADMINISTRABLE TEST,” filed May 20, 2020; U.S. Provisional Patent Application No. 63/027,874, titled “RAPID SELF ADMINISTRABLE TEST,” filed May 20, 2020; U.S. Provisional Patent Application No. 63/027,890, titled “RAPID SELF ADMINISTRABLE TEST,” filed May 20, 2020; U.S. Provisional Patent Application No. 63/027,864, titled “RAPID SELF ADMINISTRABLE TEST,” filed May 20, 2020; U.S. Provisional Patent Application No. 63/027,878, titled “RAPID SELF ADMINISTRABLE TEST,” filed May 20, 2020; U.S. Provisional Patent Application No. 63/027,886, titled “RAPID SELF ADMINISTRABLE TEST,” filed May 20, 2020; U.S. Provisional Patent Application No. 63/053,534, titled “COMPUTER VISION ALGORITHM FOR DIAGNOSTIC TESTING,” filed on Jul. 17, 2020; and U.S. Provisional Patent Application No. 63/116,603, titled “SOFTWARE ECOSYSTEM FOR HEALTH MONITORING,” filed Nov. 20, 2020, each of which is hereby incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Described herein in one embodiment is a computer-implemented method comprising accessing information representing a detection component (e.g., a test strip or colorimetric assay) of a diagnostic test and determining, based at least in part on the information representing the detection component of the diagnostic test, results of the diagnostic test. A number of exemplary diagnostic tests are provided herein that can be used with such techniques, including diagnostic tests useful for detecting target nucleic acid sequences. The tests, as described herein, are able to be performed in a point-of-care (POC) setting or home setting without specialized equipment.

In one embodiment, the information representing the detection component of the diagnostic test may comprise image data.

In one embodiment, the diagnostic test may comprise obtaining a sample from a subject, processing the sample, and analyzing the sample with the detection component.

In one embodiment, the results of the diagnostic test may comprise at least one of a diagnosis of the subject or a validity of the diagnostic test.

In one embodiment, the diagnostic test may detect a presence or absence of one or more target nucleic acids in the sample, a degree of positivity or negativity of the one or more target nucleic acids in the sample, a confidence associated with the presence or absence of the one or more target nucleic acids in the sample, or some combination thereof.

In one embodiment, the one or more target nucleic acids may represent at least one of a viral, fungal, parasitic, or protozoan pathogen.

In one embodiment, the subject may be a human being having or suspected of having a viral infection, and the diagnosis of the subject may comprise a diagnosis of the viral infection.

In one embodiment, the detection component may comprise a test strip configured to form one or more lines indicating the results of the diagnostic test.

In one embodiment, the detection component may comprise a colorimetric assay configured to form one or more colors indicating the results of the diagnostic test.

In one embodiment, determining the results of the diagnostic test may comprise processing the image data representing the detection component of the diagnostic test with a computer vision algorithm to obtain an output, and determining, based on the output of the computer vision algorithm, the results of the diagnostic test.

In one embodiment, the computer vision algorithm may comprise a trained machine learning model.

Described herein in one embodiment is a method comprising obtaining a sample from the subject, processing the sample, analyzing the sample with a detection component of the diagnostic test, and performing a computer-implemented method comprising accessing information representing the detection component of the diagnostic test, and determining, based at least in part on the information representing the detection component of the diagnostic test, results of the diagnostic test.

In one embodiment, the sample may comprise nucleic acids from the subject.

In one embodiment, processing the sample may comprise performing lysis on the sample and amplifying the nucleic acids of the sample.

In one embodiment, analyzing the sample with the detection component may comprise screening the nucleic acids for one or more target nucleic acids using the detection component.

In one embodiment, screening the nucleic acids may comprise adding the amplified nucleic acids of the sample to the detection component.

In one embodiment, the sample may comprise at least one of saliva, nasal mucus, or cellular scraping.

In one embodiment, the sample may be obtained by swabbing.

In one embodiment, the information representing the detection component of the diagnostic device comprises image data.

In one embodiment, the results of the diagnostic test may comprise at least one of a diagnosis of the subject or a validity of the diagnostic test.

In one embodiment, the diagnostic test may detect a presence or absence of one or more target nucleic acids in the sample.

In one embodiment, the one or more target nucleic acids may represent at least one of a viral, fungal, parasitic, or protozoan pathogen.

In one embodiment, the subject may be a human being having or suspected of having a viral infection, and the diagnosis of the subject may comprise a diagnosis of the viral infection.

In one embodiment, the detection component may comprise a test strip configured to form one or more lines indicating the results of the diagnostic test.

In one embodiment, the detection component may comprise a colorimetric assay configured to form one or more colors indicating the results of the diagnostic test.

In one embodiment, determining the results of the diagnostic test may comprise processing the image data representing the detection component of the diagnostic test with a computer vision algorithm to obtain an output and determining, based on the output of the computer vision algorithm, the results of the diagnostic test.

In one embodiment, the computer vision algorithm may comprise a trained machine learning model.

Described herein in one embodiment is a computer-implemented method comprising accessing image data representing a detection component of a diagnostic test, processing the image data representing the detection component of the diagnostic test with a computer vision algorithm to obtain an output, and determining, based on the output of the computer vision algorithm, the results of the diagnostic test.

In one embodiment, the diagnostic test may comprise obtaining a sample from a subject, processing the sample, and analyzing the sample with the detection component.

In one embodiment, the results of the diagnostic test may comprise at least one of a diagnosis of the subject or a validity of the diagnostic test.

In one embodiment, the diagnostic test may detect a presence or absence of one or more target nucleic acids in the sample.

In one embodiment, the one or more target nucleic acids may represent at least one of a viral, fungal, parasitic, or protozoan pathogen.

In one embodiment, the subject may be a human being having or suspected of having a viral infection, and the diagnosis of the subject may comprise a diagnosis of the viral infection.

In one embodiment, the detection component may comprise a test strip configured to form one or more lines indicating the results of the diagnostic test.

In one embodiment, the detection component may comprise a colorimetric assay configured to form one or more colors indicating the results of the diagnostic test.

In one embodiment, the computer vision algorithm may comprise a trained machine learning model.

In one embodiment, the machine learning model may comprise a convolutional neural network.

Described herein in one embodiment is a method of performing a diagnostic test on a subject, the method comprising obtaining a sample from the subject, processing the sample, analyzing the sample with a detection component of the diagnostic test, and performing a computer-implemented method comprising accessing image data representing the detection component of the diagnostic test, processing the image data representing the detection component of the diagnostic test with a computer vision algorithm to obtain an output, and determining, based on the output of the computer vision algorithm, the results of the diagnostic test.

In one embodiment, the sample may comprise nucleic acids from the subject.

In one embodiment, processing the sample may comprise performing lysis on the sample and amplifying the nucleic acids of the sample.

In one embodiment, analyzing the sample with the detection component may comprise screening the nucleic acids for one or more target nucleic acids using the detection component.

In one embodiment, screening the nucleic acids may comprise adding the amplified nucleic acids of the sample to the detection component.

In one embodiment, the sample may comprise at least one of saliva, nasal mucus, or cellular scraping.

In one embodiment, the sample may be obtained by swabbing.

In one embodiment, the data may comprise image data.

In one embodiment, the results of the diagnostic test may comprise at least one of a diagnosis of the subject or a validity of the diagnostic test.

In one embodiment, the diagnostic test may detect a presence or absence of one or more target nucleic acids in the sample.

In one embodiment, the one or more target nucleic acids may represent at least one of a viral, fungal, parasitic, or protozoan pathogen.

In one embodiment, the subject may be a human being having or suspected of having a viral infection and the diagnosis of the subject may comprise a diagnosis of the viral infection.

In one embodiment, the detection component may comprise a test strip configured to form one or more lines indicating the results of the diagnostic test.

In one embodiment, the detection component may comprise a colorimetric assay configured to form one or more colors indicating the results of the diagnostic test.

In one embodiment, the computer vision algorithm may comprise a trained machine learning model.

In one embodiment, the machine learning model may comprise a convolutional neural network.

Described herein in one embodiment is a system comprising a diagnostic test kit configured for performing a diagnostic test, the diagnostic test kit comprising a detection component, at least one computer hardware processor, and at least one non-transitory computer-readable storage medium storing processor-executable instructions that, when executed by the at least one computer hardware processor, cause the at least one computer hardware processor to perform a method comprising: accessing an image of the detection component of the diagnostic test kit and determining, based at least in part on the image of the detection component of the diagnostic test kit, results of the diagnostic test.

In one embodiment, the system further may comprise a portable electronic device; and the method further comprises directing a user of the portable electronic device to capture the image of the detection component of the diagnostic test kit with the portable electronic device.

In one embodiment, directing the user of the portable electronic device to capture the image of the detection component may comprise displaying a visual indicator via a display of the portable electronic device, the visual indicator directing the user of the portable electronic device to capture an image of the detection component of the diagnostic test kit.

In one embodiment, the diagnostic test may comprise obtaining a sample from a subject, processing the sample, and analyzing the sample with the detection component.

In one embodiment, the results of the diagnostic test may comprise at least one of a diagnosis of the subject or a validity of the diagnostic test.

In one embodiment, the diagnostic test may detect a presence or absence of one or more target nucleic acids in the sample.

In one embodiment, the one or more target nucleic acids may represent at least one of a viral, fungal, parasitic, or protozoan pathogen.

In one embodiment, the subject may be a human being having or suspected of having a viral infection, and the diagnosis of the subject may comprise a diagnosis of the viral infection.

In one embodiment, the detection component may comprise a test strip configured to form one or more lines indicating the results of the diagnostic test.

In one embodiment, the detection component may comprise a colorimetric assay configured to form one or more colors indicating the results of the diagnostic test.

In one embodiment, the method may further comprise displaying the results of the test. In one embodiment, the method may further comprise displaying the results of the test to the user via the display of the portable electronic device.

In one embodiment, the results of the diagnostic test may comprise processing the image of the diagnostic test with a computer vision algorithm to obtain an output, and determining, based on the output of the computer vision algorithm, the results of the diagnostic test.

In one embodiment, the computer vision algorithm may comprise a trained machine learning model.

In one embodiment, the portable electronic device may comprise a camera, and the image of the detection component of the diagnostic test kit may be captured using the camera of the portable electronic device.

Described herein in one embodiment is a non-transitory computer-readable media comprising instructions that, when executed by one or more processors on a computing device, are operable to cause the one or more processors to: access an image of a detection component of a diagnostic test kit for performing a diagnostic test; and determine, based at least in part on the image of the detection component of the diagnostic test kit, results of the diagnostic test.

Described herein in one embedment is a non-transitory computer-readable media comprising instructions that, when executed by one or more processors on a computing device, are operable to cause the one or more processors to: access image data representing a detection component of a diagnostic test; process the image data representing the detection component of the diagnostic test with a computer vision algorithm to obtain an output; and determine, based on the output of the computer vision algorithm, the results of the diagnostic test.

Described herein in one embedment is a method of performing a diagnostic test on a subject, the method comprising: performing a computer-implemented method comprising: accessing image data representing a detection component of a diagnostic test; processing the image data representing the detection component of the diagnostic test with a computer vision algorithm to obtain an output; and determining, based on the output of the computer vision algorithm, the results of the diagnostic test.

In an embodiment, the method further comprises obtaining a sample from the subject.

In an embodiment, the method further comprises processing the sample.

In an embodiment, the method further comprises analyzing the sample with the detection component of the diagnostic test.

### BRIEF DESCRIPTION OF THE DRAWINGS

FIGS. 1A-1B show screenshots from an exemplary companion mobile application (app), including a “Record Results” screen, “Image Acquisition” screen, “Test Complete” screen for negative test result, and “Test Complete” screen for positive test result;

FIG. 2 shows an exemplary lateral flow strip key for interpreting results, including the key itself, a positive test, and a negative test;

FIG. 3A illustrates an exemplary method for determining diagnostic test results, according to some embodiments;

FIG. 3B illustrates an exemplary trained machine learning model for determining diagnostic test results, according to some embodiments;

FIG. 4 depicts an illustrative implementation of a computer system that may be used in connection with some embodiments of the technology described herein;

FIG. 5 shows, according to some embodiments, a detection component comprising a “chimney”;

FIG. 6 shows a diagnostic kit comprising a sample-collecting component, a reaction tube, a detection component, and a heater, according to some embodiments;

FIG. 7 shows, according to some embodiments, a cartridge comprising a first reservoir, a second reservoir, a third reservoir, a vent path, a detection region, and a pumping tool;

FIG. 8 shows, according to some embodiments, a diagnostic kit comprising a sample-collecting component and a cartridge; and

FIG. 9 shows, according to some embodiments, a diagnostic device comprising a plurality of blister packs.

### DETAILED DESCRIPTION

### I. Machine Vision Techniques

The present disclosure provides systems and methods for automatically determining the results of a diagnostic test using machine vision techniques. In some embodiments, the techniques include using image processing techniques (e.g., computer vision techniques) to automatically determine the results based on an image of a detection component of the diagnostic test.

The machine vision techniques can be used with any diagnostic test. An exemplary diagnostic test with which the machine vision technique can be used is described herein. That exemplary diagnostic test can detect one or more target nucleic acid sequences (e.g., a nucleic acid sequence of a pathogen, such as SARS-CoV-2 or an influenza virus). A diagnostic system may comprise a sample-collecting component (e.g., a swab) and a diagnostic device (e.g., a cartridge, a blister pack, and/or a “chimney” detection device, as described herein). A diagnostic test may include steps of collecting a sample from a subject (e.g., a human being, such as a patient being tested for a disease), processing the sample (e.g., with any of the processing techniques described herein), and analyzing the sample with a detection component (e.g., a lateral flow assay strip, a colorimetric assay). For example, a colorimetric assay or test strip, such as a lateral flow test strip, may be used to analyze the sample and indicate, via lines on the test strip, colors on the colorimetric assay, or any other suitable indicator, visual information regarding the results of the test.

The inventors have recognized and appreciated that interpreting the visual information indicated on the detection component of a diagnostic test can present a challenge with conventional techniques. In general, with conventional techniques, a human observer (e.g., a doctor, nurse, or other medical professional) may determine the results of the diagnostic test based on the visual information of the diagnostic test detection component. Human error in interpreting the visual information indicated on the detection component of the diagnostic test can lead to errors in determining the test results. For example, a user of a diagnostic test may misread the test as positive when it in fact it is negative or invalid.

A particular challenge recognized and appreciated by the inventors is that the user of the diagnostic test may be unable to read the test result and/or may incorrectly determine the test result. For example, in some cases, the diagnostic test user may be an individual without medical training (e.g., an individual who is not a nurse, doctor, or other expert) and/or an individual without sufficient training with the diagnostic test (e.g., a clinician, including a nurse, doctor, or other expert, who is not familiar with the diagnostic test). This may occur, for example, in the context of self-administered or at-home diagnostic tests, which may be carried out without the presence of a medical professional. Without medical training, the user of the diagnostic test may be unable to interpret the visual information indicated on the detection component, or may do so with decreased accuracy, confidence, and/or speed relative to a medical professional. Additionally or alternatively, this can occur in the clinical setting, when a clinician administers the test but is unable to interpret visual information indicated on the detection component (e.g., due to insufficient training of the clinician, etc.). Users may be unable to read the test for various other reasons, such as users that may have vision problems, poor lighting and/or other environmental conditions (e.g., direct sunlight if administered outdoors), intellectual issues, unintentional confusion, and so on.

The inventors have further recognized and appreciated that, in some cases, the visual information indicated on the detection component of the diagnostic may be less visible or clear than desired (e.g., lines on a test strip may be faint or blurred, the colors of the detection component may be difficult to distinguish). In some cases, it may be difficult or impossible for a human to perceive some or all of the visual information indicated on the diagnostic test detection component, resulting in reduced the accuracy of the corresponding test results. For example, in the case of a lateral flow control test strip, a user may mistakenly identify the test results as a false negative if a line indicating a positive result is faded, blurred, or otherwise difficult to perceive.

Recognizing and appreciating the foregoing, the inventors have developed machine vision techniques, including computer vision techniques, for processing image data representing detection components of diagnostic tests to obtain corresponding test results. As described herein, these techniques may include a method comprising accessing image data (e.g., stored as pixel values or in any other suitable format) representing a detection component of a diagnostic test (e.g., a lateral flow control test strip, colorimetric assay, test line(s), dyes in the testing component, light indicator(s) (e.g., LED(s)), and/or other readout component or device that provides test result information) and determining, based at least in part on the image data representing the detection component of the diagnostic test, results of the diagnostic test (e.g., a diagnosis of the patient, such as a positive or negative test result for one or more diseases of interest; a validity of the test, such as a valid or invalid test result). In some embodiments, determining the results of the diagnostic test may comprise processing the image data representing the detection component of the diagnostic test with a computer vision algorithm (e.g., a line detection algorithm, an edge detection algorithm, a convolution-based algorithm, a machine learning algorithm, or any other suitable algorithm) to obtain an output, and determining the results of the diagnostic test based on the output of the computer vision algorithm.

The machine vision techniques described herein can use machine learning models that are trained using sets of training data, which can be large, to determine the ultimate parameters of the model (e.g., of a neural network) that in-turn allow the trained neural network to perform its associated function (e.g., to identify test components in the images, determine the test result, etc.). Such machine learning models have a massive number of parameters, such as hundreds of thousands of parameters, millions of parameters, tens of millions of parameters, and/or hundreds of millions of parameters as described herein. As a result, and as described further herein, the trained neural networks can perform tasks in an automated and repeatable manner, and with high accuracy that may not otherwise be achievable through human analysis of the detection components.

As described herein, image data is not limited to pixel values or image data captured with a camera. Instead, image data may include any information representing characteristics or changes in characteristics of the readout of the diagnostic test. For example, the information may represent any combination of visual characteristics (e.g., color, intensity, texture, pattern, or shape) and/or other characteristics (e.g., chemical characteristics) of the diagnostic test detection component. As also described herein, the techniques are not limited to processing image data. The techniques can include processing non-image data instead of and/or in conjunction with image data. For example, the detection component can provide sound(s), haptic feedback, and/or electronic data through wired/wireless communication channels. The techniques can include analyzing such information, whether visual or not, to determine the test result.

In some embodiments, information representing the detection component of the diagnostic test, such as image data, may be provided directly as input to a computing device (e.g., a processor local to or integrated with the diagnostic test, or an external computing device). For example, the information representing the detection component of the diagnostic test may be provided as input to an algorithm being executed on the computing device, such as a computer vision algorithm. In some embodiments, information representing the detection component may be stored (e.g., in a local or external data store) prior to, during, or after being processed by a computer vision algorithm. In some embodiments, the techniques may comprise directing a user (e.g., a medical professional administering the diagnostic test, a user self-administering the diagnostic test) to capture an image of the detection component of the diagnostic test kit with the camera of a portable electronic device (e.g., a smartphone, cellphone, tablet). In some embodiments, the techniques may comprise displaying the results of the diagnostic test to the user (e.g., via a screen or other display of the portable electronic device).

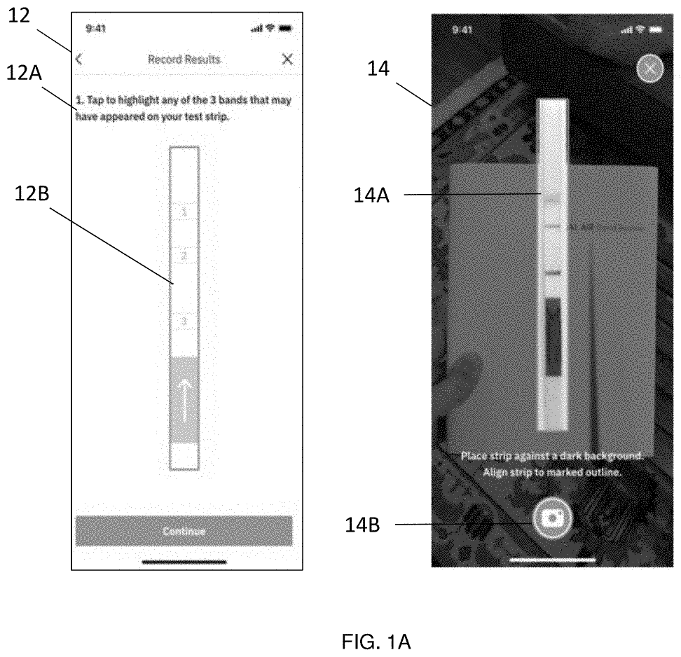

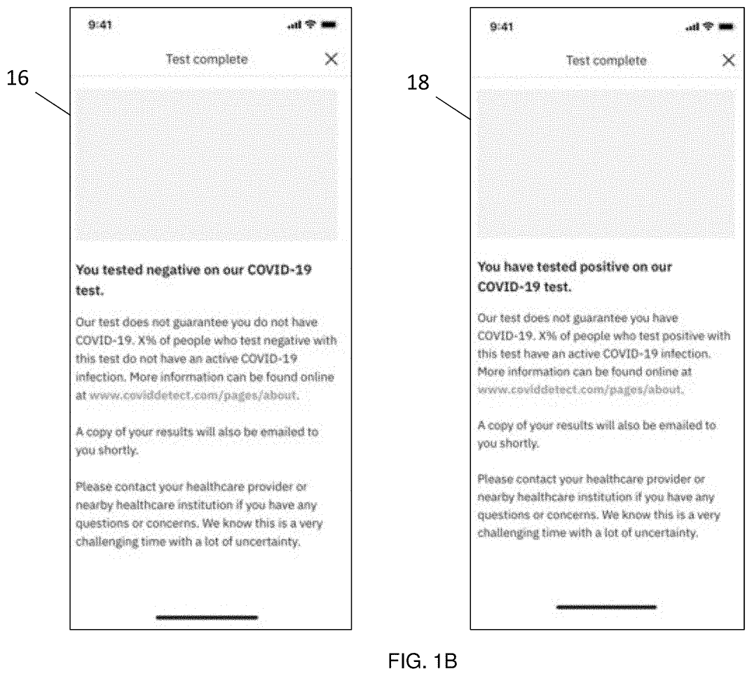

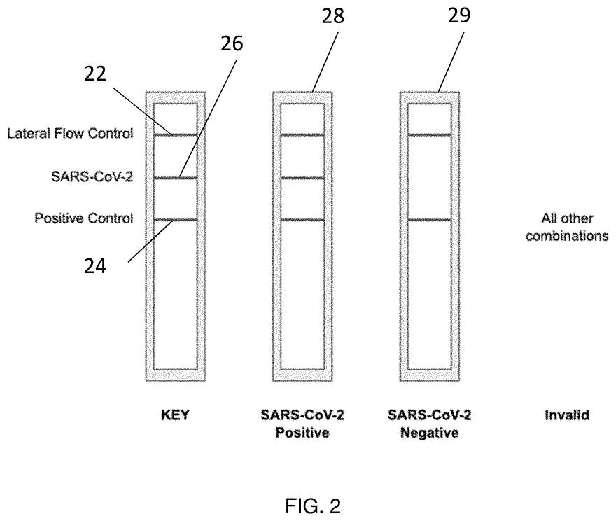

In some embodiments, a diagnostic test comprises or is associated with software to read and/or analyze test results. Such an embodiment of the software application for reporting and analyzing results is illustrated in FIGS. 1A-1B and FIG. 2. As shown, in an embodiment, the detection component includes a lateral flow control line 22, a positive control line 24, and a test target (e.g., SARS-CoV-2, influenza, other pathogen) line 26. In this example, the lateral flow control line 22 and positive control line 24 are to illustrate whether the test was performed and accomplished accurately. For example, only if both control lines 22, 24 are positive and dark (highlighted) was the test performed and accomplished accurately. In this example, if either control line 22, 24 is not positive and dark, then the test is invalid and must be repeated. In this example, if all three lines are positive and dark is the test positive (e.g., for COVID-19, influenza, or some other target nucleic acid). Only if all three lines are positive and dark is the test positive (for COVID-19, influenza, or some other target nucleic acid), as illustrated by positive reading 28. If the test target line 26 is not positive and dark, then the test is negative as illustrated by negative test reading 29. In some embodiments, the test can be guided by a software application that guides a user through steps to administer the rapid test. Examples of such software applications, as well as a software-based ecosystem for use with a rapid test, are described in co-owned U.S. patent application entitled “SOFTWARE-BASED ECOSYSTEM FOR USE WITH A RAPID TEST,” also filed on Mar. 16, 2020, which is hereby incorporated by reference herein in its entirety and referred to further herein as the “Downloadable Software and Software-Based Ecosystem Application.”

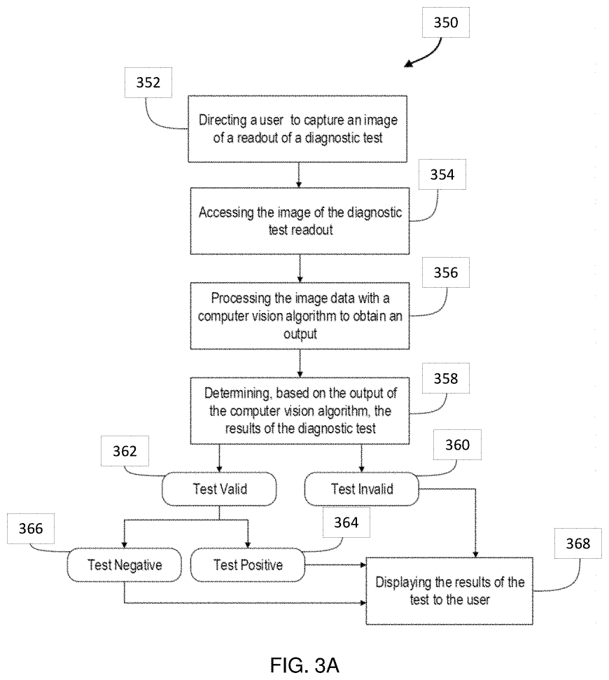

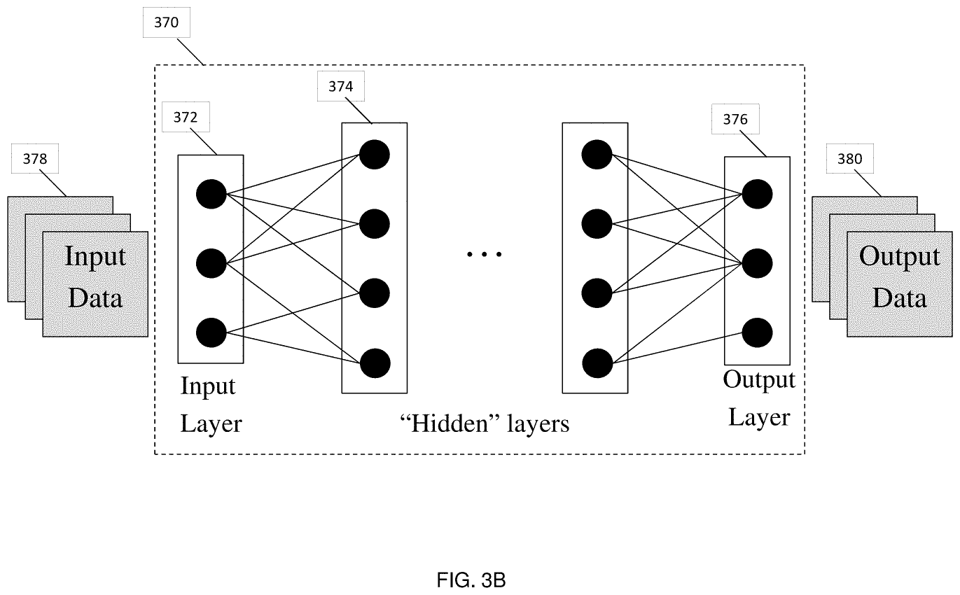

The results test can be read, read by another, or uploaded to a device containing the software application for automatic reading, as described herein at least with respect to FIGS. 1A-1B and FIG. 3A-3B. In the example of FIGS. 1A-1B, through an image appearing on a device containing the software application, a user can tap the number of lines (bands) appearing positive on the detection component (e.g., the readout strip) and the software application will automatically read the results. This is shown in the image 12 in FIG. 1A. Alternatively, a user may take an image of the detection component (e.g., the readout strip) and upload that image to the device containing the software application for automatic reading of the test results. This image taking application is illustrated in the image 14 in FIG. 1A.

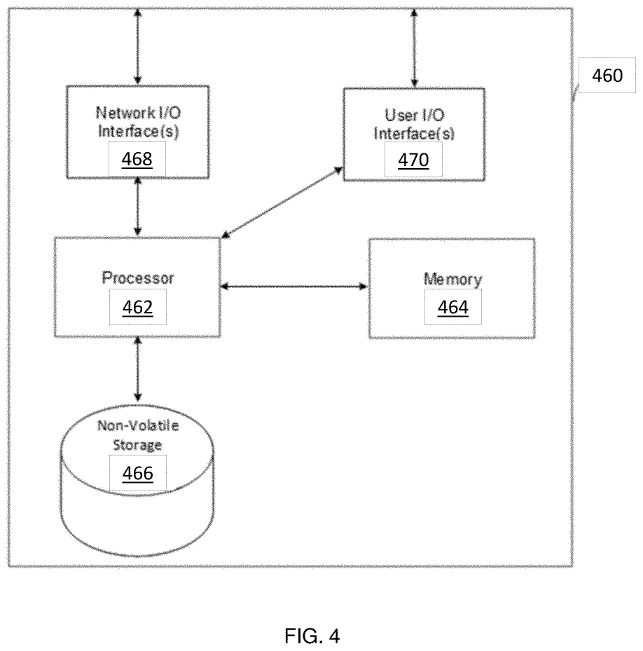

In some embodiments, a device (e.g., a camera, a smartphone) is used to generate an image of a test result (e.g., one or more lines detectable on a lateral flow assay strip). In certain cases, a machine vision software application is employed to evaluate the image and provide a positive or negative test result. The test result can be displayed, as shown by images 16 (negative result) and 18 (positive result) in FIG. 1B. FIG. 3A illustrates an exemplary method 350 for determining diagnostic test results, according to some embodiments. The acts of method 350 may be performed with respect to any of the diagnostic tests and/or test kits described herein, or any other suitable diagnostic tests and/or test kits. The acts of method 350 may be a carried out by one or more computer hardware processors, as described herein with respect to FIG. 4.

Method 350 may begin at act 352 with directing a user (e.g., a medical professional administering the test, a patient self-administering the test, or any other individual) to capture an image of a detection component of a diagnostic test. For example, the user may be directed to use a portable electronic device (such as a smartphone, tablet, or any other suitable device) to capture an image of the detection component of the diagnostic test. In some embodiments, a user can be directed to provide a representation of the detection component. For example, a user may be directed to highlight or circle aspects of a graphical representation of the detection component (e.g., a location of the detection component in a graphical representation of the detection component displayed to the user). As another example, a user may be directed to tap the locations of particular elements of the detection component in the graphical representation of the detection component (e.g., the locations of test control lines). As a result, a user need not necessarily capture an image, and can additionally or alternatively provide information about the detection component for analysis.

Directing the user to capture an image of the detection component may comprise displaying a message, alert, or interface on a display the portable electronic device. For example, as shown in image 12 of FIG. 1A, an image capturing interface of the portable electronic device may be overlaid with additional information directing the user to capture the image. This information may include text directing the user to capture the image and/or visual indicators such as shading or an outline of the detection component to be captured in the image. In FIG. 1A, for example a marked outline 14A is displayed on the portable electronic device to help the user align the image to the detection component of the diagnostic test prior to capture as shown in image 14. In some embodiments, the user may capture the photo by selecting a button (e.g., the camera icon 14B shown in image 14 in FIG. 1A). In some embodiments, the image may be captured automatically (e.g., when the detection component is aligned with a corresponding outline, or when the detection component is automatically detected in the image).

In some embodiments, directing the user to capture an image of the detection component may comprise automatically guiding the user to capture the image (e.g., via the portable electronic device). For example, the portable electronic device may provide audio or visual feedback to the user indicating that an acceptable image of the detection component has been captured (e.g., by displaying a check mark or other indication of success, highlighting an outline of the detection component in a captured image, or any other suitable indication of success). Additionally or alternatively, the portable electronic device may provide feedback while directing the user to capture an acceptable image of the detection component (e.g., by displaying an arrow, a moving outline of the detection component, an indication of image quality, a progress bar, or any other suitable audio or visual indicator). In some embodiments, the portable electronic device can automatically capture the image. In some embodiments, the portable electronic device can monitor the data provided by the imaging device and determine when the portable electronic device is able to capture a sufficient image of the detection component. For example, the portable electronic device can perform image processing techniques to analyze data received from the imaging device to determine whether (or not) a detection component is in the field of view of the portable electronic device. In some embodiments, the portable electronic device can perform, for example, feature matching, pattern matching, character recognition, and/or other machine vision techniques to determine when the detection component is sufficiently in the field of view of the imaging device. Upon such a determination, the portable electronic device can automatically capture one or more images of the detection component.

In some embodiments, the user may utilize an interface of the portable electronic device to specify further information about the detection component in the captured image. For example, the user may be directed to highlight or circle a location of the detection component in the image, and/or may be directed to tap the locations of particular elements of the detection component in the image (e.g., the locations of lines appearing on a test strip). In the image 12 of FIG. 1A, for example, a text message 12A and visual indicator 12B are displayed that direct the user to tap to highlight any of three bands appearing on an exemplary test stip.

In some embodiments, act 352 may further comprises storing the image (e.g., in a data store internal to the portable electronic device capturing the image) and/or transmitting the image (e.g., to an external data store or server, via any suitable connection for transmitting, such as a wired or wireless network connection). The image may be stored and/or transmitted automatically (e.g., without any human input), or may be stored and/or transmitted manually (e.g., with a human providing input, such as with a user interface, to indicate location(s) at which the image is to be stored and/or destination(s) to which the image is to be sent). The image may be stored and/or transmitted in any suitable format. For example, the image may be represented and stored as pixel values (e.g., RGB values and/or intensity values), or any other suitable representation of the image data, in any suitable data structure (e.g., an array).

The method 350 may continue at act 354 with accessing the image of the detection component of the diagnostic test. For example, the image may be stored in a data store associated with the portable electronic device and accessed from that data store at act 354. Alternatively or additionally, the image data may be received or accessed from an external data store or server. In some embodiments, rather than beginning at act 352, the method 350 may begin at act 354 with the image data being accessed (e.g., the image data may be received from an external source or accessed from an internal or external data store, without needing to be captured by a user). In some embodiments, the image data may be accessed directly from the detection component. For example, the diagnostic test itself may be configured to capture (e.g., with suitable hardware or software elements of the diagnostic test) image data representing the detection component. The image data may be stored, accessed, and/or processed locally (e.g., using a storage medium and/or processor associated with the diagnostic test) or may be transmitted (e.g., to an external data store or server, such as described above with respect to act 352) for further processing. In some embodiments, only a portion of the image data may be accessed. For example, the image may be automatically cropped (e.g., by selectively removing, masking, or otherwise ignoring portions of the image data) so as to retain only image data regarding an area of interest (e.g., the area of the image including the detection component).

The method 350 may continue at act 356 with processing the image data with a computer vision algorithm to obtain an output. As described herein at least with respect to act 352 and act 354, the image data may be processed in any suitable format (such as pixel values in an array) and provided as one or more inputs to the computer vision algorithm. In some embodiments, the computer vision algorithm may include one or more of a line detection algorithm or an edge detection algorithm (e.g., a Hough transform, a Canny edge detector, or any other suitable technique for line or edge detection). In some embodiments, the computer vision algorithm may include performing feature extraction on the image (e.g., by applying an unsupervised learning technique to the image, or using any other suitable techniques for feature extraction). In some embodiments, the computer vision algorithm may include convolution-based techniques (e.g., including convolution filters which may be applied to pixels of the input image). In some embodiments, the computer vision algorithm may include comparing lines and/or other markings that appear in the image with known patterns of lines and/or markings.

In some embodiments, the computer vision algorithm may include a machine learning model. For example, the computer vision algorithm may include a machine learning model comprising a neural network. In some embodiments, the computer vision algorithm may comprise a neural network having multiple layers (e.g., at least two layers, at least five layers, or at least ten layers) including one or more different types of layers (e.g., convolutional layers, feed-forward layers, pooling layers, dropout layers, or reduction layers). FIG. 3B illustrates an exemplary machine learning model 370 for determining diagnostic test results, according to some embodiments. The machine learning model 370 is a neural network that includes an input layer 372, one or more hidden layers 374 (e.g., convolutional layers), and an output layer 376. The input data 378 is provided to the machine learning model 370 and processed by the machine learning model to generate the output data 380. While FIG. 3B illustrates a neural network, it should be appreciated that any machine learning model for processing images can be used with the techniques described herein. For example, the convolutional neural network model can include a neural network model with a “U” shape, such as a U-Net architecture, and/or any other architecture sufficient for processing the image data. It should be appreciated that the computer vision techniques described herein can include machine learning techniques, but are not so limited and may additionally and/or alternatively include other machine vision algorithms that do not use machine learning techniques.

As described herein, the input data 378 can be an image of a detection component. In some embodiments, the input data 378 is preprocessed prior to being provided to the machine learning model 370. For example, the image (e.g., of the detection component) can be augmented using one or more transformations, such as rotation, scaling, elastic transformations, Gaussian blur adjustments, intensity changes, cropping, and/or reflection. Other pre-processing can be used as well. For example, the image can be preprocessed by performing an object recognition process that identifies one or more objects in the image (e.g., control lines) and annotates the identified objects for processing by the machine learning model 370.

Regardless of which computer vision techniques are employed as part of processing the image at act 356, the output data (e.g., output data 380 in FIG. 3B) may include information relating to the detection component of the diagnostic test appearing in the image. For example, the output of the computer vision algorithm may identify a location of the detection component in the image (e.g., with a bounding box or mask). The output of the computer vision algorithm may additionally or alternatively identify locations of features of the detection component, such as lines (e.g., a lateral flow control line, a SARS-CoV-2 line, and/or a positive control line) or other indicators (e.g., dark portions, colors, etc.) which may or may not be visible to a human observer. The output of the computer vision algorithm may additional or alternatively include information such as color/intensity information, a confidence score, or any other information regarding the detection component of the diagnostic test. In some embodiments, if the output of the computer vision algorithm differs from a user input (e.g., an input provided via a user interface, as described above with respect to act 352) the user may be notified of the difference, and/or prompted (e.g., by the portable electronic device) to confirm that the user input provided was accurate (e.g., by selecting a button or providing other suitable input indicating confirmation). In some embodiments, for example when the output of the computer vision algorithm differs from a user input, the user may be prompted to repeat act 352 and/or provide additional user input (e.g., to highlight or circle a location of the detection component in the image or tap the locations of particular elements of the detection component in the image).

The neural network model may be a trained neural network model. For example, the neural network model may be trained on training data including training images (e.g., images of diagnostic test detection components) which may comprise hundreds, thousands, tens of thousands, or more images. According to some embodiments, the training data may be augmented with transformations or otherwise pre-processed as part of training the neural network as described herein. Each training image can be associated with ground truth data indicative of the test result associated with the image and/or other aspects of the detection component that can be used to determine the test result. For example, each training image can be labeled and/or otherwise associated with the results of the diagnostic test. As another example, the training images can be associated with labels that identify a location of detection component(s) in the image. As a further example, the training images can include data indicative of locations of features of the detection component, such as lateral flow lines and/or other indicators (e.g., dark portions, colors, etc.) which may or may not be visible to a human observer. As an additional example, the images may be annotated with color/intensity information and/or any other information regarding the detection component of the diagnostic test.

According to some embodiments, the training data can be built over time to add training data as it becomes available (e.g., new test data, test results, etc.). In some embodiments, new models can be trained using the updated and/or most recent training data (or subsets thereof, as desired, such as using some of the data set for training and some of the training set for validation, etc.). In some embodiments, already trained models can be updated and/or further trained using new training data as it becomes available. In some embodiments, different models can be trained for different types of tests. For example, one model can be trained for lateral flow tests, another model can be trained for colorimetric assay tests, and/or the like. In some embodiments, a single model can be trained for multiple types of tests. For example, different tests can use lateral flow test strips but provide different results (e.g., through the activation of different test and/or control lines). A single model can be trained to categories the different types of tests and/or test results. Additionally, or alternatively, a single model can be trained for different types of tests (e.g., for test strip-based tests and colorimetric-based tests).

The machine learning models are trained using such training data to determine the ultimate parameters of the neural network, which allow the trained neural network to automatically determine the output data that provides and/or is used to determine the test result. Such machine learning models can have a massive number of parameters. For example, machine learning models can have at least half a million parameters, one million parameters, or more. In some embodiments, such models can include tens of millions of parameters (e.g., ten million parameters, twenty-five million parameters, fifty million parameters, or more). In some embodiments, the parameters can include at least a hundred million parameters (e.g., at least one hundred million parameters, between one million to one hundred million parameters), hundreds of million parameters, at least a billion parameters, and/or any suitable number or range of parameters. Training the neural network with training data to learn the parameters results in trained machine learning models that can perform tasks in an automated and repeatable manner, and with high accuracy. In particular, since the trained neural networks are trained using large sets of training data, the models are sufficiently robust so as not to be affected by the environment in which the images were captured (e.g., lighting, temperature, humidity, etc.), image noise (e.g., resulting from the environmental conditions and/or other conditions, such as background objects, hand shaking, images only capturing a portion of the detection component, etc.), and/or any other differences that may occur among images being analyzed using the techniques described herein.

The method 350 may continue at act 358 with determining, based on the output of the computer vision algorithm, the results of the diagnostic test. As shown in the figure, determining the results of the diagnostic test based on the output of the computer vision algorithm may comprise determining whether the results of the test are invalid 360 or valid 362. For example, in the case of a detection component shown in FIGS. 1A-1B and FIG. 2, the absence of a positive control line indicates that the diagnostic test is invalid. In this example, if the computer vision algorithm output indicates that there is no positive control line on the detection component, then the result of the diagnostic test may be considered invalid 360. Otherwise, if the positive control line is indicated in the computer vision algorithm output, then the result of the test may be considered valid 362. In some embodiments, features other than lines (e.g., colors or other visual indicators) may be used to determine the validity or invalidity of the test based on the output of the computer vision algorithm.

In some embodiments the method 350 may further include determining whether the results of the test are positive 364 or negative 366. According to some embodiments, rather than first determining the validity or invalidity of the diagnostic test result, the method may directly check whether the computer vision algorithm output is indicative of a positive or negative result and thereby infer whether result is valid (e.g., the detection component corresponds to a known positive or negative result, such as in the examples of FIGS. 1A-1B and FIG. 2). In some cases, the results may include multiple validity, invalidity, positive, and/or negative results (e.g., for diagnostic tests that test for multiple diseases). In some embodiments, the method 350 may additionally or alternatively include determining a confidence of the test result and/or a degree of positivity or negativity of the test result. For example, the method 350 may provide a degree of positivity or potential positivity. As another example, the method 350 may provide a “positive” or “negative” result and an associated confidence of the result (e.g., 100% confidence, 99% confidence, 95% confidence, 80% confidence, etc.). As a further example, the method 350 can provide an indication that the test result is inconclusive. For example, if the method 350 has a confidence below a threshold percentage (e.g., below 80%, below 70%, below 50%), then instead of providing a positive or negative result, the method can instead indicate that the test is inconclusive.

Regardless of the results determined at act 358, the method 350 may continue at act 368 with displaying the results of the diagnostic test to the user. As discussed herein, the results of the test, once determined according to method 350 or by any other means, may be communicated directly to the user or directed to another, such as a medical professional. For example, the results of the diagnostic test may be visually displayed to the user via the portable electronic device used to capture the image of the diagnostic test. The images 16 and 18 of FIG. 1B provide an example of diagnostic test results on a display of a portable electronic device. As shown in the figure, the test result may include textual information including links (e.g., links to further information), data regarding the accuracy of the test, or any other relevant information. In some embodiments, the user may be shown a corresponding “Test Complete” screen on the portable electronic device, which may tell the user if the test result is positive, negative, or invalid. In addition to providing the test result, careful language may be used to ensure that the user can properly interpret the meaning of the result. Additionally or alternatively, the results of the diagnostic test may be communicated to the user via other means, such as an audible signal (e.g., spoken words, or a chime, tone, beep, or other noise which may be played, for example, from the portable electronic device).

In some embodiments, the results of the diagnostic test may be communicated to the user via electronic mail, text message, telephone call, physical mail, or any other suitable means of communication. In some embodiments, the results of the diagnostic test may be accessed via an application, such as an application of the portable electronic device or a web application. Accessing the results of the diagnostic test may include requiring the user to verify their identity, such as by providing credentials (e.g., a username and password, biometric information, or other suitable identification). Additionally or alternatively, in some embodiments, the results of the diagnostic test may be transmitted (e.g., via a wired or wireless network connection) to a computing device (e.g., a smart phone, one or more processors arranged in a cloud computing configuration, or any other suitable computing device) for processing.

In some embodiments, a software-based testing ecosystem can be used to provide a user access to test results and/or to receive health information about a patient, including disease or antibody test data. The testing ecosystem can integrate various data to provide a central medical and testing resource for users to track disease progression. The testing ecosystem can integrate data provided by users and/or data that is obtainable from other resources. In some embodiments, the data integrated into the ecosystem includes user information, account information, medical records, rapid test data, other testing data (e.g., antibody tests and/or other viral, bacterial, fungal, parasitic and/or protozoan pathogen tests), and/or the like. In some embodiments, the patient information includes at least one of name, social security number, date of birth, address, phone number, email address, medical history, and medications. In an embodiment, the data can be obtained from resources such as clinician databases, medical record databases, agency databases, and/or any other resources with relevant data.

In some embodiments, the software-based testing ecosystem includes one more compute resources and/or databases to store test results and patient information. The testing ecosystem can store the information in a central database and/or send the information to one or more other locations (e.g., clinicians, authorities, etc.). In an embodiment, the software application sends test information (e.g., test readings and/or information) to a secure, HIPAA-compliant, cloud-based software infrastructure of the software ecosystem. The software ecosystem can therefore facilitate simple, fast, and scalable reporting to the federal and state health agencies. Further examples of such a software ecosystem are provided in the Downloadable Software and Software-Based Ecosystem Application reference above and incorporated herein.

In some embodiments, acts of method 350 may be omitted, repeated, performed in parallel, or otherwise altered in sequence from the example shown in FIG. 3A. For example, the acts relating to capturing the image data may be omitted in some embodiments (e.g., when previously captured image data is received from an external source, rather than being captured on the same device performing the other acts of method 350). In some embodiments, act 368 may be omitted (e.g., if the results are to be transmitted to a remote server, viewed by a medical professional, or otherwise processed other than by displaying them to a user).

That results of the test, once determined according to method 350 or by any other means, may be communicated directly to the user or directed to another, such as a medical professional. The test results can be communicated to a central database server and/or to a remote doctor or other. A remote server having a database is envisioned to store test results and may as well store user or patient information. In some embodiments, the central database also stores patient information. In some embodiments, the patient information is an electronic medical record. In some embodiments, the patient information includes at least one of name, social security number, date of birth, address, phone number, email address, medical history, and medications.

In some embodiments, the remote database server may track and monitor locations of users (e.g., using smartphones or remote devices with tracking capabilities). In some cases, the remote database server can be used to notify individuals who come into contact with or within a certain distance of any user who has tested positive for a particular illness (e.g., COVID-19). In some cases, a user's test results, information, and/or location may be communicated to state and/or federal health agencies. The locations may also be communicated to a central database server and/or to a remote doctor or other.

In other embodiments, the mobile application may send this information (e.g., an image of the resultant lateral flow test strip) to a secure, HIPAA-compliant, cloud-based software infrastructure. This software infrastructure may then facilitate simple, fast, and scalable reporting to the federal and state health agencies.

In some embodiments, the database may generate a code based on the user's results (e.g., positive or negative for the viral illness). After a successful test, the code is available in the application. In some embodiments, the code is read by a bar code scanner or other security detection device. If the user is negative for the viral illness and has a negative code, the security system will recognize the code and permit entry. In other embodiments, if the user is positive for the viral illness and has a positive code, the security system will recognize the code and deny entry.

It should be appreciated that while embodiments described herein provide for using computer vision techniques to automatically determine the results of a diagnostic test based on an image of a detection component, the techniques are not so limited. In some embodiments, the detection component may provide non-visual test result information in addition to and/or instead of visual information. For example, the non-visual test result information can include audible tone(s), haptic feedback, computerized information (e.g., data packets sent over wired and/or wireless transmission links), electronic signals (e.g., current applied to one or more terminals that can be monitored for test results), and/or any other information that can be used to provide data associated with a test result.

The computer vision techniques described herein can be used alone and/or in conjunction with other automated techniques used to analyze non-visual test result information to determine the results of a diagnostic test. In some embodiments, a device (e.g., a camera, a smartphone) is used to detect and process the non-visual information. For example, in some embodiments one or more microphones are used to detect one or more audible sounds provided by the detection component, and the device analyzes the sound(s) to automatically determine the results of the diagnostic test. For example, the techniques can include analyzing the tone, frequency, pitch, and/or other aspects of the sounds to determine the test result.

As another example, in some embodiments the device analyzes computerized test data determine the results of the diagnostic test. The computerized test data can be accessed (e.g., accessed from a computer memory, either as part of the diagnostic test and/or as part of a computing device automatically determining the diagnostic test result) and/or received by the device via wired and/or wireless communication channel(s) with the diagnostic test. The data may include data indicative of aspects of the test, such as whether one or more control lines were activated (or not), how much the one or more control lines were activated (e.g., fully activated, partially activated, etc.), and/or the like. The device can include one or more algorithms configured to process the electronic data to determine the test result.

It should be appreciated that the test result can therefore be provided using visual information and/or non-visual information, and the techniques can be configured to process such information as necessary to automatically determine the result of the test.

II. Exemplary Tests for Use with the Machine Vision Techniques

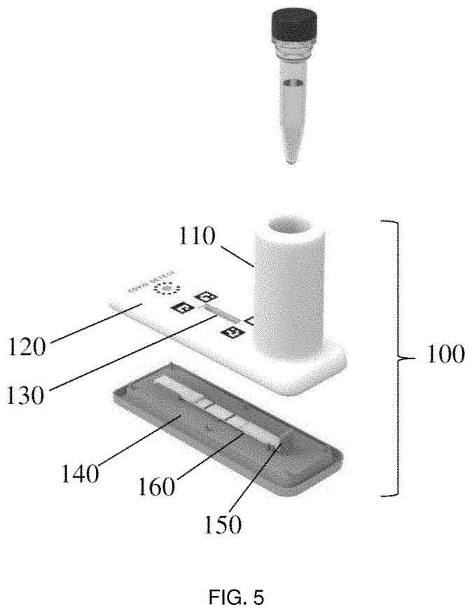

The following sections describe aspects of exemplary diagnostic devices, tests and test steps, including in conjunction with FIGS. 5-9 that can be used with the machine learning techniques described herein, which are for illustrative purposes and are not intended to be limiting. Therefore, it should be appreciated that the machine learning techniques described herein are not limited to such aspects, and can be used with any test, diagnostic device, or test kit.

Diagnostic devices, systems, and methods described herein may be safely and easily operated or conducted by untrained individuals (e.g., untrained clinicians, at-home users, etc.). Unlike prior art diagnostic tests, some embodiments described herein may not require knowledge of even basic laboratory techniques (e.g., pipetting). Further, due to the rapid spread and evolution diseases, it is desirable to quickly administer tests in a manner that does not necessarily require training to understand how to read and/or interpret the test results. Therefore, the machine learning techniques described herein can be provided in conjunction with these and other tests in order to provide for rapid deployment and use of tests.

As a result, the diagnostic devices, systems, and methods described herein may be useful in a wide variety of contexts. For example, in some cases, the diagnostic devices and systems may be available over the counter for use by consumers. In such cases, untrained users may be able to administer the diagnostic test. In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist and/or traveling clinician).

A. Target Nucleic Acid Sequences

The diagnostic devices, systems, and methods, in some embodiments, may be used to detect the presence or absence of any target nucleic acid sequence (e.g., from any pathogen of interest, cancer cell, etc.). Target nucleic acid sequences may be associated with a variety of diseases or disorders. In some embodiments, the diagnostic devices, systems, and methods are used to diagnose at least one disease or disorder caused by a pathogen (e.g., a viral, bacterial, fungal, protozoan, or parasitic pathogen). In some embodiments, the diagnostic devices, systems, and methods are configured to identify particular strains of a pathogen (e.g., a virus). In certain embodiments, a diagnostic device comprises a lateral flow assay strip comprising one or more test lines. In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2, SARS-CoV-2 D614G, an influenza type A virus, and/or an influenza type B virus).

B. Overview of Exemplary Diagnostic Systems

According to some embodiments, diagnostic systems comprise a sample-collecting component (e.g., a swab) and a diagnostic device. In certain cases, the diagnostic device comprises a cartridge (e.g., a microfluidic cartridge), a blister pack, and/or a “chimney” detection component. In some cases, the diagnostic device comprises a detection component (e.g., a lateral flow assay strip, a colorimetric assay). In certain embodiments, the diagnostic device further comprises one or more reagents (e.g., lysis reagents, nucleic acid amplification reagents, CRISPR/Cas detection reagents). In some instances, at least one reagent is contained within the diagnostic device (e.g., within a cartridge, a blister pack, and/or a “chimney” detection component) of a diagnostic system. In certain other embodiments, the diagnostic system separately includes one or more reaction tubes comprising the one or more reagents. Each of the one or more reagents may be in liquid form (e.g., in solution) or in solid form (e.g., lyophilized, dried, crystallized, air jetted). The diagnostic device may also comprise an integrated heater, or the diagnostic system may comprise a separate heater.

C. Lysis Reagents

A lysis reagent generally refers to a reagent that promotes cell lysis either alone or in combination with one or more reagents and/or conditions (e.g., heating). In some cases, the one or more lysis reagents comprise one or more enzymes, detergents, and/or RNase inhibitors (e.g., a murine RNase inhibitor). In some embodiments, the one or more reagents comprise one or more reagents to reduce or eliminate potential carryover contamination from prior tests (e.g., prior tests conducted in the same area). In certain embodiments, the one or more reagents comprise one or more reverse transcription reagents.

In some embodiments, the one or more reagents comprise one or more nucleic acid amplification reagents. A nucleic acid amplification reagent generally refers to a reagent that facilitates a nucleic acid amplification method. In some embodiments, the nucleic acid amplification method is an isothermal nucleic acid amplification method. In some cases, an isothermal nucleic acid amplification method, unlike PCR, avoids use of expensive, bulky laboratory equipment for precise thermal cycling. Non-limiting examples of suitable isothermal nucleic acid amplification methods include loop-mediated isothermal amplification (LAMP), recombinase polymerase amplification (RPA), nicking enzyme amplification reaction (“NEAR”), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In certain embodiments, the one or more nucleic acid amplification reagents comprise LAMP reagents, RPA reagents, or NEAR reagents.

D. Detection Components

In certain embodiments, the diagnostic device (e.g., cartridge, blister pack, “chimney” detection component) comprises a detection component. In some embodiments, the detection component comprises a lateral flow assay strip or a colorimetric assay. Examples of both are provided herein. In some embodiments, results of the lateral flow assay strip and/or colorimetric assay are read and/or analyzed by software (e.g., a mobile application).

In some embodiments, the diagnostic device comprises a lateral flow assay strip. In some embodiments, the lateral flow assay strip is configured to detect one or more target nucleic acid sequences. In certain cases, the lateral flow assay strip comprises one or more fluid-transporting layers comprising one or more materials that allow fluid transport (e.g., via capillary action). In some embodiments, the one or more fluid-transporting layers of the lateral flow assay strip comprise a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action).

In certain embodiments, the lateral flow assay strip comprises one or more sub-regions. A first sub-region (e.g., a sample pad) can be where a fluidic sample is introduced to the lateral flow assay strip. A second sub-region (e.g., a particle conjugate pad) can include a plurality of labeled particles. A third sub-region (e.g., a test pad) can include one or more test lines. In some embodiments, a first test line comprises a capture reagent (e.g., an immobilized antibody) configured to detect a first target nucleic acid sequence. In certain embodiments, the lateral flow assay strip comprises one or more additional test lines. In some instances, each test line of the lateral flow assay strip is configured to detect a different target nucleic acid. A fourth sub-region (e.g., a wicking area) can absorb fluid flowing through the lateral flow assay strip. In some embodiments, the diagnostic device comprises a plurality of lateral flow assay strips.

In some embodiments, the diagnostic device comprises a colorimetric assay. In certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers. In some cases, a colorimetric reaction may occur in each peripheral chamber as described herein, resulting in varying colors in the peripheral chambers.

The diagnostic system, in some embodiments, comprises a heater. In certain embodiments, the heater is integrated with the diagnostic device. In some embodiments, the diagnostic system comprises a separate heater (i.e., a heater that is not integrated with other system components). In some cases, the heater comprises a battery-powered heat source, a USB-powered heat source, a hot plate, a heating coil, and/or a hot water bath.

E. “Chimney” Detection Component Embodiments

In some embodiments, a diagnostic device comprises a detection component comprising a “chimney” configured to receive a reaction tube. In certain embodiments, the “chimney” detection component comprises a lateral flow assay strip as described herein with one or more test lines configured to detect one or more target nucleic acid sequences (e.g., and one or more control lines). One embodiment of a “chimney” detection component is shown in FIG. 5. In FIG. 5, detection component 100 comprises chimney 110, front panel 120 comprising opening 130, and back panel 140 comprising puncturing component 150 and lateral flow assay strip 160. In some embodiments, chimney 110 and front panel 120 are integrally formed. In operation, a reaction tube comprising fluidic contents may be inserted into chimney 110. As shown in FIG. 5, the bottom end of the reaction tube is inserted into chimney 110. The reaction tube may be punctured by puncturing component 130. As a result, at least a portion of the fluidic contents of the reaction tube may be deposited on a first sub-region (e.g., a sample pad) of lateral flow assay strip 160 and transported through lateral flow assay strip 160 (e.g., via capillary action).

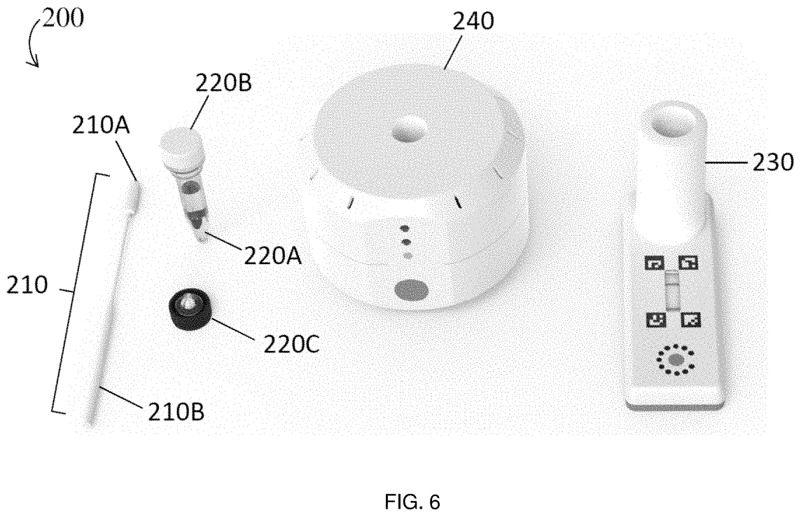

One embodiment of a diagnostic system comprising a “chimney” detection component is shown in FIG. 6. In FIG. 6, diagnostic system 200 comprises sample-collecting component 210, reaction tube 220, “chimney” detection component 230, and heater 240. As shown in FIG. 6, sample-collecting component 210 may be a swab comprising swab element 210A and stem element 210B. In certain embodiment, reaction tube 220 comprises tube 220A, first cap 220B, and second cap 220C (e.g., with one or more reagents and/or a reaction buffer). In operation, a user may collect a sample that is inserted into the fluidic contents of tube 220A similar as described above. In some embodiments, reaction tube 220 may be inserted into heater 240 and heated. Following heating, reaction tube 220 may be inserted into “chimney” detection component 230 such that at least a portion of the fluidic contents of reaction tube 220 are deposited onto a portion of a lateral flow assay strip of “chimney” detection component 230.

F. Cartridge Embodiments

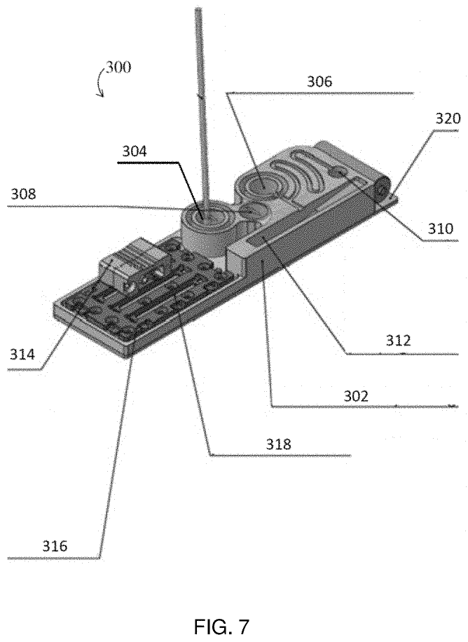

In some embodiments, a diagnostic device comprises a cartridge (e.g., a microfluidic cartridge). An exemplary cartridge 300 is shown in FIG. 7, which comprises cartridge body 302. As shown in FIG. 7, cartridge body 302 comprises first reagent reservoir 304, second reagent reservoir 306, third reagent reservoir 308, vent path 310, and detection region 312. The detection region 312 can include a lateral flow assay strip configured to detect one or more target nucleic acid sequences. In certain embodiments, the lateral flow assay strip comprises one or more test lines comprising one or more capture reagents (e.g., immobilized antibodies) configured to detect one or more target nucleic acid sequences as described herein.

In operation, a user may use a swab to collect a sample from a subject and then expose the contents of first reagent reservoir 304. In some embodiments, chemical lysis may be performed by one or more lysis reagents (e.g., enzymes, detergents) in first reagent reservoir 304. In some embodiments, the user may push pumping tool 314 along pump lanes, including to cause the fluidic contents of second reagent reservoir 306 (e.g., amplicon-containing fluid) to be transported from second reagent reservoir 306 to detection region 312. In some cases, the fluidic contents may flow through the lateral flow assay strip (e.g., via capillary action). In some cases, at least a portion of the lateral flow assay strip may be visible to the user, and the user may be able to determine whether or not one or more target nucleic acid sequences are present based on the formation (or lack thereof) of one or more opaque lines (or other markings) on the lateral flow assay strip.

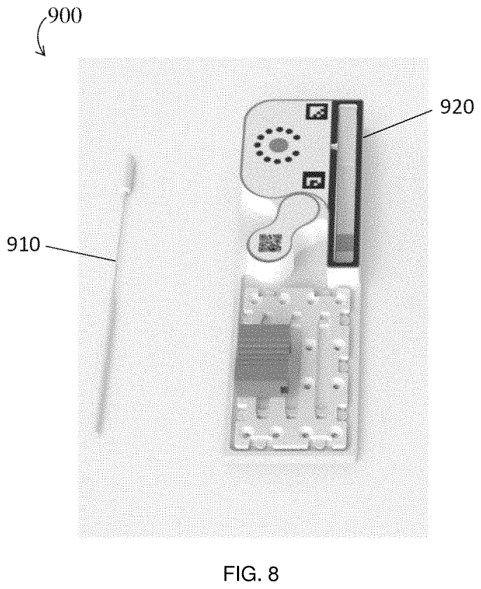

In some cases, a cartridge may be a component of a diagnostic system. For example, FIG. 8 illustrates an exemplary diagnostic system 900 comprising sample-collecting swab 910 and cartridge 920. In some embodiments, the diagnostic system may be used with an electronic device (e.g., a smartphone, a tablet) and associated software (e.g., a mobile application). In certain embodiments, for example, the software may provide instructions for using the cartridge, may read and/or analyze results, and/or report results. In certain instances, the electronic device may communicate with the cartridge (e.g., via a wireless connection).

G. Blister Pack Embodiments

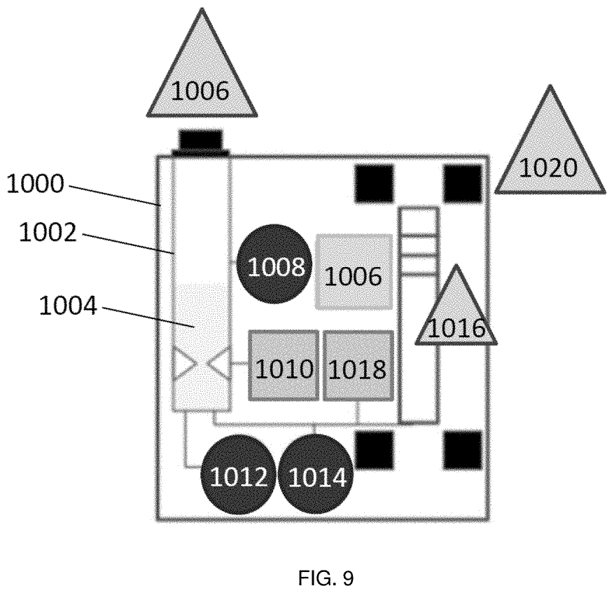

In some embodiments, a diagnostic device comprises one or more blister packs. One embodiment is shown in FIG. 9. In FIG. 9, diagnostic device 1000 comprises tube 1002 containing reaction buffer 1004. In certain embodiments, diagnostic device 1000 comprises a heater in thermal communication with tube 1002.

In operation, a sample may be added through sample port 1006. A first blister pack 1008 comprising one or more lysis and/or decontamination reagents (e.g., UDG) are released from blister pack 1008 into tube 1002. In some embodiments, tube 1002 may be heated by a heater (not shown in FIG. 9). In some cases, mechanism 1010 provides a physical mechanism to reduce sample volume as needed. In certain embodiments, one or more amplification reagents are released from amplification blister pack 1012 into tube 1002. In some instances, a dilution buffer may optionally be released from dilution blister pack 1014 into tube 1002. The sample is then flowed across a lateral flow assay strip 1016, with mechanism 1018 ensuring that the sample accesses lateral flow assay strip 1016 at the appropriate time (e.g., after the processing is complete). In certain embodiments, one or more markers 1020 (e.g., one or more ArUco markers) are provided to facilitate image alignment and. The lateral flow strip may indicate whether one or more target nucleic acid sequences are present in the sample. In some embodiments, the results on the lateral flow strip are interpreted using a mobile software-based application, downloadable to a smart device, such as that described herein.

H. Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time.

I. Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulose, protease, and glycanase. In some embodiments, the one or more lysis reagents comprise one or more detergents.

J. Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. As described herein, the nucleic acid amplification reagents can be LAMP reagents, RPA reagents, and or agents for NEAR reactions.

1. Loop-Mediated Isothermal Amplification (LAMP)

In some embodiments, the DNA sample is subjected to loop-mediated isothermal amplification (LAMP) instead of RPA. In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

In some embodiments, the LAMP reagents comprise a FIP and a BIP for one or more target nucleic acids. In some embodiments, the LAMP reagents comprise an F3 primer and a B3 primer for one or more target nucleic acids. In some embodiments, the LAMP reagents comprise a forward loop primer and a backward loop primer for one or more target nucleic acids. In some embodiments, the control nucleic acid is a nucleic acid sequence encoding human RNase P. In some embodiments, one or more LAMP primers comprise a label. In some embodiments, the LAMP reagents comprise a DNA polymerase with high strand displacement activity. In some embodiments, the LAMP reagents comprise deoxyribonucleotide triphosphates (“dNTPs”). In some embodiments, the LAMP reagents comprise magnesium sulfate (MgSO4). In some embodiments, the LAMP reagents comprise betaine.

For example, a biotinylated FIP primer is incubated with the nucleic acid sample (e.g., DNA) for 30 minutes at 65° C. Then, a specific FITC-labeled probe is added to the reaction mixture and incubated for another 10 minutes at 65° C., resulting in a dual-labeled LAMP product. Then, detection buffer containing rabbit anti-FITC antibodies coupled to colloidal gold is mixed with the reaction mixture, and the lateral flow test strip is inserted into the tube. In a positive reaction, the double labeled LAMP product migrates with the buffer flow and is retained at the test line by a biotin ligand present on the test line. The gold coupled anti-FITC antibody binds to the FITC molecule at the probe, and an opaque band develops over time. In a negative sample, the reactions do not occur, and no opaque band develops in the test line. The control line comprises an anti-rabbit antibody, which retains some of the unbound gold-conjugated antibody, resulting in an opaque band in the control line.

In one embodiment, the nucleic sample is subjected to colorimetric LAMP. A gold- or antigen-labeled probe is added to the sample. If the probes bind their target, then the labeled probes are dispersed throughout the solution during the reaction (resulting in one color). If, however, the probes do not bind their target, they aggregate instead, resulting in a second color. By reading the different colors of the test, a user can determine whether a sample is positive or negative for a target sequence (e.g., COVID19).

K. Molecular Switches

As described herein, a sample undergoes lysis and amplification prior to detection. The reagents associated with lysis and/or detection may be in solid form (e.g., lyophilized, dried, crystallized, air jetted). In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat.

L. Detection Processes

In some embodiments, amplified nucleic acids (i.e., amplicons) may be detected using any suitable methods. In some embodiments, one or more target nucleic acid sequences are detected using a lateral flow assay strip. In some embodiments, one or more target nucleic acid sequences are detected using a colorimetric assay.

In some embodiments, one or more target nucleic acid sequences are detected using a lateral flow assay strip (e.g., in a “chimney” detection component, a cartridge, a blister pack, as described herein). In some embodiments, a fluidic sample (e.g., fluidic contents of a reaction tube, a reagent reservoir, and/or a blister pack chamber) is transported through the lateral flow assay strip via capillary action.

As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin.

### III. Computer Implementation

An illustrative implementation of a computer system 460 that may be used in connection with any of the embodiments of the technology described herein (e.g., such as the method of FIG. 3A and/or the machine learning model of FIG. 3B) is shown in FIG. 4. The computer system 460 includes one or more processors 462 and one or more articles of manufacture that comprise non-transitory computer-readable storage media (e.g., memory 464 and one or more non-volatile storage media 466). The processor 462 may control writing data to and reading data from the memory 464 and the non-volatile storage device 466 in any suitable manner, as the aspects of the technology described herein are not limited in this respect. To perform any of the functionality described herein, the processor 462 may execute one or more processor-executable instructions stored in one or more non-transitory computer-readable storage media (e.g., the memory 464), which may serve as non-transitory computer-readable storage media storing processor-executable instructions for execution by the processor 462.

Computing device 460 may also include a network input/output (I/O) interface 468 via which the computing device may communicate with other computing devices (e.g., over a network), and may also include one or more user I/O interfaces 470, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description, but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

What is claimed is:

1. A computer-implemented method comprising:
accessing information representing a detection component of a diagnostic test; and
determining, based at least in part on the information representing the detection component of the diagnostic test, results of the diagnostic test.

2. The computer implemented method of claim 1, wherein the information representing the detection component of the diagnostic test comprises image data.

3. The computer-implemented method of claim 1, wherein the diagnostic test detects a presence or absence of one or more target nucleic acids in the sample, a degree of positivity or negativity of the one or more target nucleic acids in the sample, a confidence associated with the presence or absence of the one or more target nucleic acids in the sample, or some combination thereof.

4. The computer-implemented method of claim 1, wherein:
the diagnostic test comprises:
obtaining a sample from a subject;
processing the sample; and
analyzing the sample with the detection component; and
the results of the diagnostic test comprise at least one of a diagnosis of the subject or a validity of the diagnostic test.

5. The computer-implemented method of claim 1, wherein the detection component comprises a test strip configured to form one or more lines indicating the results of the diagnostic test.

6. The computer-implemented method of claim 1, wherein the detection component comprises a colorimetric assay configured to form one or more colors indicating the results of the diagnostic test.

7. The computer-implemented method of claim 1, wherein determining the results of the diagnostic test comprises:
processing the image data representing the detection component of the diagnostic test with a computer vision algorithm to obtain an output; and
determining, based on the output of the computer vision algorithm, the results of the diagnostic test.

8. The computer-implemented method of claim 7, wherein the computer vision algorithm comprises a trained machine learning model.

9. A system comprising:
at least one computer hardware processor; and
at least one non-transitory computer-readable storage medium storing processor-executable instructions that, when executed by the at least one computer hardware processor, cause the at least one computer hardware processor to perform a method comprising:
accessing an image of a detection component of a diagnostic test kit for performing a diagnostic test; and
determining, based at least in part on the image of the detection component of the diagnostic test kit, results of the diagnostic test.

10. The system of claim 9, wherein:
the system comprises a portable electronic device; and
the method further comprises directing a user of the portable electronic device to capture the image of the detection component of the diagnostic test kit with the portable electronic device.

11. The system of claim 10, wherein directing the user of the portable electronic device to capture the image of the detection component comprises:
displaying a visual indicator via a display of the portable electronic device, the visual indicator directing the user of the portable electronic device to capture an image of the detection component of the diagnostic test kit.

12. The system of claim 9, wherein:
the results of the diagnostic test comprise at least one of a diagnosis of the subject or a validity of the diagnostic test.

13. The system of claim 9, wherein the detection component comprises a test strip configured to form one or more lines indicating the results of the diagnostic test.

14. The system of claim 9, wherein the detection component comprises a colorimetric assay configured to form one or more colors indicating the results of the diagnostic test.

15. The system of claim 9, wherein the method further comprises:
displaying the results of the test.

16. The system of claim 9, wherein the method further comprises:
displaying the results of the test to the user via a display.

17. The system of claim 9, wherein determining the results of the diagnostic test comprises:
processing the image of the diagnostic test with a computer vision algorithm to obtain an output; and
determining, based on the output of the computer vision algorithm, the results of the diagnostic test.

18. The system of claim 17, wherein the computer vision algorithm comprises a trained machine learning model.

19. The system of claim 9, wherein the system comprises a portable electronic device comprising a camera, and the image of the detection component of the diagnostic test kit is captured using the camera of the portable electronic device.

20. A non-transitory computer-readable media comprising instructions that, when executed by one or more processors on a computing device, are operable to cause the one or more processors to:
access an image of a detection component of a diagnostic test kit for performing a diagnostic test; and
determine, based at least in part on the image of the detection component of the diagnostic test kit, results of the diagnostic test.
